(19) United States
(12) Patent Application Publication
(10) Pub. No.: US 2026/0363635 A1
(43) Pub. Date: Nov. 19, 2026

(54) MEDICAL DEVICE WITH IMPROVEMENT ALARM MODE

(71) Applicant: Welch Allyn, Inc., Skaneateles Falls, NY (US)

(72) Inventors: Hannah Drum Paquette, Savannah, GA (US); Kathryn M. Coles, Syracuse, NY (US); Lauren N. Hayward, San Diego, CA (US); WonKyung McSweeney, Manilus, NY (US); Thomas A. Myers, Syracuse, NY (US); Rebecca Quilty-Koval, Baldwinsville, NY (US)

(21) Appl. No.: 19/673,090

(22) Filed: May 11, 2026

Related U.S. Application Data
(60) Provisional application No. 63/804,775, filed on May 13, 2025.

(51) Int. Cl. A61B 5/00; A61B 5/0205

(52) U.S. Cl. CPC A61B 5/746; A61B 5/0205; A61B 5/7275; A61B 5/7435; A61B 2560/0276

(57) ABSTRACT

A medical device displays a trend visualization showing measured values of a physiological variable relative to default alarm limits. The medical device detects an alarm condition when the measured values exceed a default upper alarm limit or are less than a default lower alarm limit. The medical device displays setup windows that guide clinical workflow for initiating an improvement alarm mode including: a first window showing suggested starting alarm limits based on recent measurements of the physiological variable; a second window for selecting an expiration time for the improvement alarm mode; and a third window summarizing the suggested starting alarm limits and the expiration time. Upon activation of the improvement alarm mode, the medical device displays a visualization showing the measured values relative to automatically adjusting alarm limits that begin with the starting alarm limits and gradually increase or decrease to follow a predicted improvement curve.

### CROSS-REFERENCE TO RELATED APPLICATIONS

This application claims the benefit of U.S. Provisional Patent Application No. 63/804,775, filed May 13, 2025, the disclosure of which is hereby incorporated by reference in its entirety.

### BACKGROUND

Nuisance alarms in medical devices are false or non-urgent alerts that can cause unnecessary disruptions in healthcare environments. These alarms, while designed to notify healthcare providers of potential issues, often arise from settings that are too sensitive, improper configurations, or routine fluctuations that do not require immediate attention. The constant barrage of nuisance alarms can lead to alarm fatigue, where healthcare professionals may begin to overlook or ignore alarms, potentially jeopardizing patient safety. Addressing nuisance alarms is crucial in improving the efficiency and effectiveness of medical device systems, ensuring that only critical alerts grab the attention of healthcare professionals.

### SUMMARY

In general terms, the present disclosure relates to medical device alarm management. In one possible configuration, a medical device is configurable to setup and initiate an improvement alarm mode for execution on the medical device. Various aspects are described in this disclosure, which include, but are not limited to, the following aspects.

One aspect relates to a medical device comprising: at least one processing device communicatively coupled to a display and to at least one sensor that captures measurements of a physiological variable; and a memory device storing instructions which, when executed by the at least one processing device, cause the at least one processing device to: display on the display a trend visualization showing measured values of the physiological variable relative to default alarm limits that include a default upper alarm limit and a default lower alarm limit; detect an alarm condition when the measured values exceed the default upper alarm limit or when the measured values are less than the default lower alarm limit; display setup windows that guide clinical workflow for initiating an improvement alarm mode, the setup windows including: a first window showing suggested starting alarm limits based on recent measurements of the physiological variable, the starting alarm limits including at least one of a starting upper alarm limit differing from the default upper alarm limit and a starting lower alarm limit differing from the default lower alarm limit; a second window for selecting an expiration time for the improvement alarm mode; and a third window summarizing the suggested starting alarm limits and the expiration time for the improvement alarm mode before activation; and upon activation of the improvement alarm mode, display a visualization showing the measured values relative to automatically adjusting alarm limits that begin with the starting alarm limits and gradually increase or decrease to follow a predicted improvement curve.

Another aspect relates to a method of managing alarms on a medical device, the method comprising: displaying a trend visualization showing values of a physiological variable relative to default alarm limits including a default upper alarm limit and a default lower alarm limit; detecting an alarm condition when the values exceed the default upper alarm limit or when the values are less than the default lower alarm limit; displaying setup windows that guide clinical workflow for initiating an improvement alarm mode, the setup windows including: a first window showing suggested starting alarm limits based on recent measurements of the physiological variable, the starting alarm limits including at least one of a starting upper alarm limit differing from the default upper alarm limit and a starting lower alarm limit differing from the default lower alarm limit; a second window for selecting an expiration time for the improvement alarm mode; and a third window summarizing the suggested starting alarm limits and the expiration time selected for the improvement alarm mode before activation; and upon activation of the improvement alarm mode, displaying a visualization showing the values of the physiological variable relative to automatically adjusting alarm limits that begin with the starting alarm limits and gradually increase or decrease to follow a predicted improvement curve.

Another aspect relates to a medical device comprising: at least one processing device communicatively coupled to a display and to at least one sensor that captures measurements of a physiological variable; and a memory device storing instructions which, when executed by the at least one processing device, cause the at least one processing device to: generate an alarm when the measurements of the physiological variable are higher than a default upper alarm limit or when the measurements of the physiological variable are lower than a default lower alarm limit; display a user interface having an input to configure an improvement alarm mode; upon selection of the input to configure the improvement alarm mode, display a first window having a starting upper alarm limit that differs from the default upper alarm limit and a starting lower alarm limit that differs from the default lower alarm limit; upon acceptance of the starting upper and lower alarm limits, display a second window displaying one or more expiration times for the improvement alarm mode; upon selection of an expiration time for the improvement alarm mode, display a third window summarizing the starting upper and lower alarm limits accepted in the first window and the expiration time selected in the second window, the third window including an input to initiate the improvement alarm mode; and upon selection of the input to initiate the improvement alarm mode, execute the improvement alarm mode for the physiological variable, wherein the improvement alarm mode automatically adjusts the starting upper and lower alarm limits toward the default upper and lower alarm limits.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of a medical device.

FIG. 2 is a rear isometric view of the medical device of FIG. 1.

FIG. 3 is a front view of the medical device of FIG. 1.

FIG. 4 is a bottom view of the medical device of FIG. 1.

FIG. 5 schematically illustrates an example of the medical device of FIG. 1.

FIG. 6 schematically illustrates an example of a method of managing alarms on the medical device of FIG. 1.

FIG. 7 illustrates an example of a home screen user interface that can be displayed on a display of the medical device of FIG. 1.

FIG. 8 illustrates an example of an alarm settings user interface displayed on the display of the medical device of FIG. 1 in response to receiving a tactile input on an alarm tab in the home screen user interface of FIG. 7.

FIG. 9 illustrates an example of a first setup window that can be displayed on the display of the medical device of FIG. 1 as a result of selecting a set improvement mode icon in the alarm settings user interface of FIG. 8.

FIG. 10 illustrates an example of a second setup window that can be displayed on the display of the medical device of FIG. 1 as a result of selecting an accept icon in the first setup window shown in FIG. 9.

FIG. 11 illustrates an example of a third setup window that can be displayed on the display of the medical device of FIG. 1 as a result of selecting a next icon in the second setup window shown in FIG. 10.

FIG. 12 illustrates another example of a home screen user interface that can be displayed on the display of the medical device of FIG. 1 after initiation of an improvement alarm mode configured using the setup windows of FIGS. 9-11.

FIG. 13 illustrates another example of an alarm settings user interface displayed on the display of the medical device of FIG. 1 in response to receiving a tactile input on an alarm tab in the home screen user interface of FIG. 12.

FIG. 14 illustrates another example of an alarm settings user interface displayed on the display of the medical device of FIG. 1 after the improvement alarm mode has been initiated on the medical device of FIG. 1.

FIG. 15a illustrates another example of a visualization that can be displayed in the alarm settings user interface of FIG. 14.

FIG. 15b illustrates another example of a visualization that can be displayed in the alarm settings user interface of FIG. 14.

FIG. 16 illustrates an example of a configuration user interface that can be displayed on the display of the medical device of FIG. 1 to configure an improvement mode tool.

### DETAILED DESCRIPTION

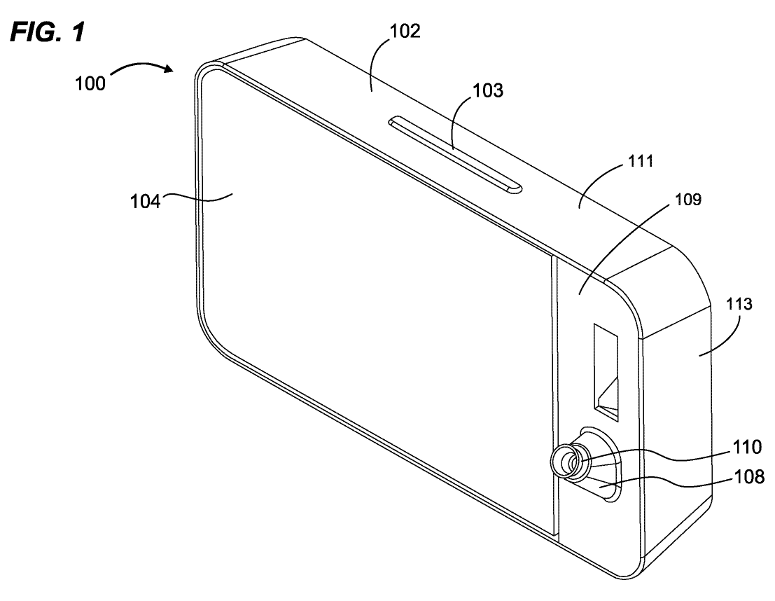

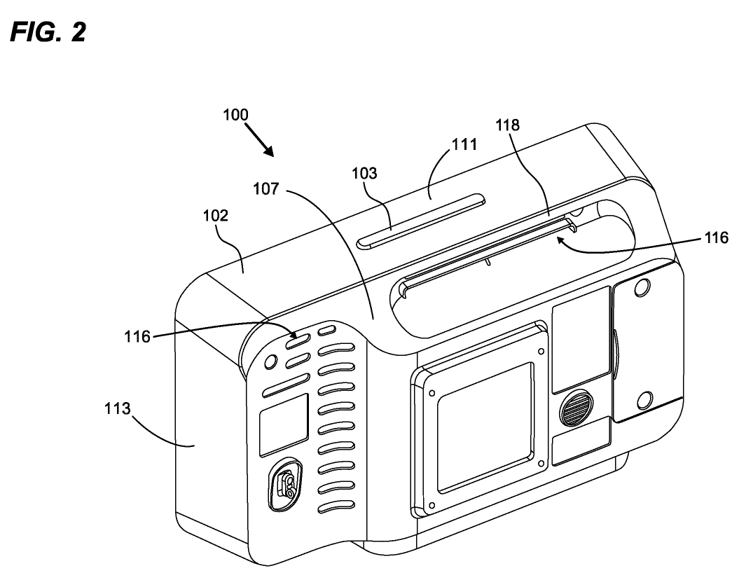

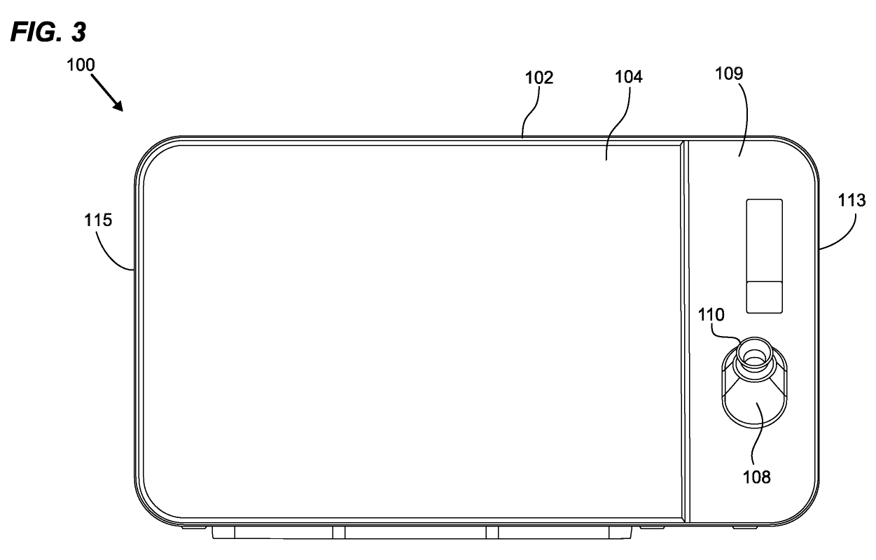

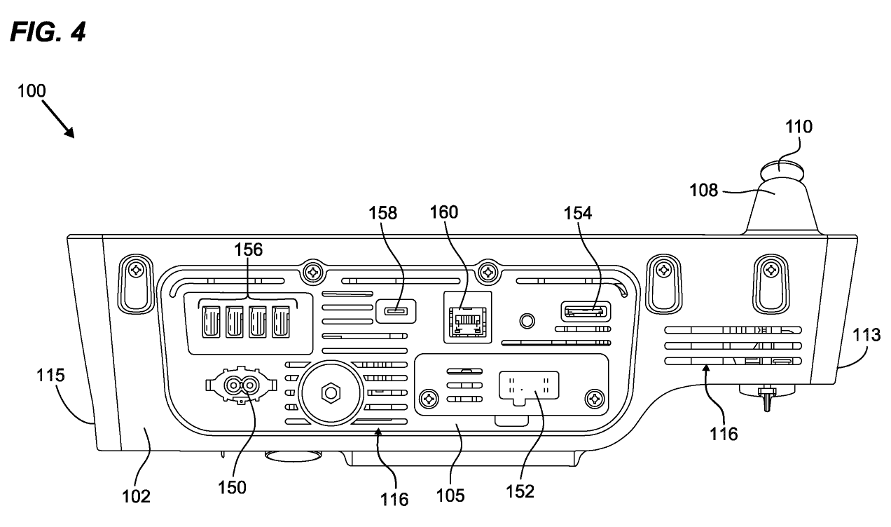

FIG. 1 is a front isometric view of a medical device 100 for monitoring one or more physiological variables of a patient. FIG. 2 is a rear isometric view of the medical device 100. FIG. 3 is a front view of the medical device 100. FIG. 4 is a bottom view of the medical device 100. As shown in FIGS. 1-4, the medical device 100 includes a housing 102 having a bottom surface 105, a rear surface 107, a front surface 109, a top surface 111, and lateral sides 113, 115 which give the medical device 100 a substantially rectangular prism shape. The industrial design shown in FIGS. 1-4 is illustrative and may vary in different examples of the medical device 100.

In some examples, the medical device 100 is portable. In such examples, the medical device 100 can include a handle 118 (see FIG. 2) that allows the housing 102 to be gripped with one hand for physically moving the medical device 100 from one location to another.

A display 104 is mounted on a front surface 109 of the housing 102. As will be described further below, the display 104 is a touchscreen that operates to both display data captured by one or more sensors communicatively coupled to the medical device 100 and to receive inputs from a user such as to adjust one or more alarm parameters and/or to silence an alarm. The housing 102 can also support physical push buttons to receive inputs from the user.

The medical device 100 is designed for use in a clinical setting. For example, the medical device 100 can be used to continuously monitor one or more physiological variables of a patient admitted to a medical facility such as a hospital, a medical clinic, and the like. Additionally, the medical device 100 can operate as a spot monitor for episodic monitoring of one or more physiological variables of a plurality of patients in the medical facility.

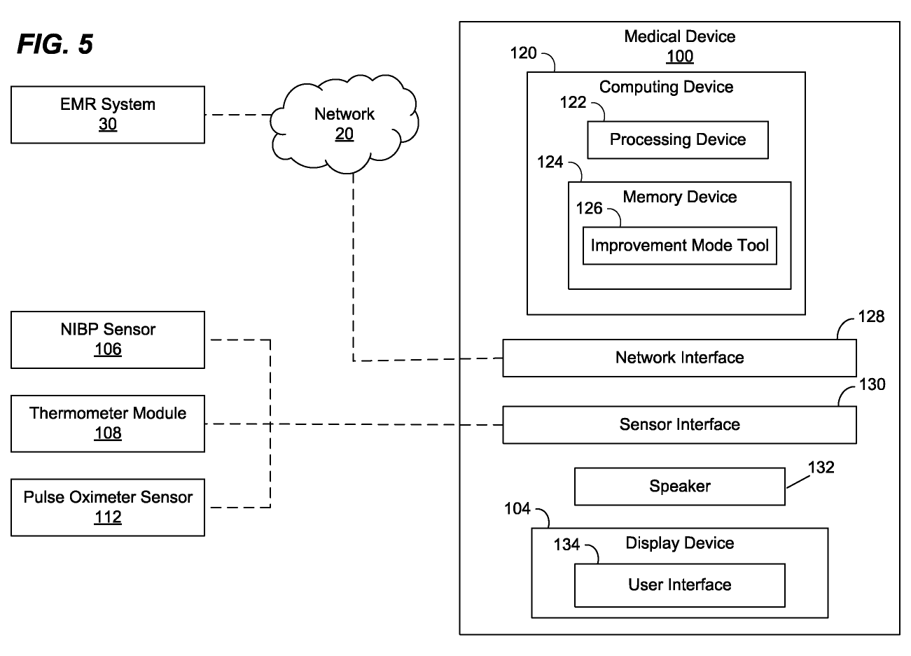

The medical device 100 is configured for connection to one or more sensors (see FIG. 5) that can be used to measure one or more physiological variables including, without limitation, non-invasive systolic and diastolic blood pressure, blood oxygen saturation (SpO2), respiration rate, pulse rate, and body temperature. For example, as shown in FIGS. 1, 3, and 4, the sensors can include a thermometer module 108 that can be used to measure body temperature.

In the example shown in FIGS. 1, 3, and 4, the thermometer module 108 is integrated with the medical device 100. The thermometer module 108 includes a port 110 for housing a handheld probe that can be orally inserted into the mouth of a patient to take a temperature reading. Alternatively, the housing 102 of the medical device 100 can be configured to house a handheld probe that can be inserted into an ear of the patient to take a temperature reading.

As shown in FIG. 5, which will be described in more detail further below, the sensors can include a non-invasive blood pressure (NIBP) sensor 106 that includes a cuff that can be inflated around the arm of a patient to non-invasively measure systolic and diastolic blood pressure. The sensors can further include a pulse oximeter sensor 112 that can attach to a finger of the patient to measure vital signs including one or more of blood oxygen saturation (SpO2), pulse rate, and respiration rate. It is contemplated that the medical device 100 can interface with additional types of sensors for measuring additional types of physiological variables.

The sensors can include cables that terminate into connectors that plug into various ports included on the housing 102 of the medical device 100 for transmission of data to the medical device 100. As shown in the example of FIG. 4, the housing 102 includes a first port 150 for connecting the NIBP sensor 106 and a second port 152 for connecting the pulse oximeter sensor 112. The medical device 100 can include a third port 154 for connecting the medical device 100 to a printer. Also, the medical device 100 can include one or more universal serial bus (USB) ports 156, a USB-C port 158, and a registered jack (RJ) interface 160. In some examples, at least some of the sensors wirelessly transmit data to the medical device 100 via a wireless connection established through Wi-Fi, Bluetooth, or another wireless protocol.

In some examples, a scanner can be connected to the medical device 100 such as by plugging into a USB port 156. The scanner can be used to scan machine-readable data such as barcodes, quick-response (QR) codes, radio-frequency identification (RFID), and the like that identify a person such as a patient who is being monitored by the medical device 100 or a user of the medical device 100 (e.g., a nurse, a doctor, and the like). The machine-readable data can also identify an object such as medical equipment or a medication being administered to the patient.

The medical device 100 receives data from the sensors for processing. The medical device 100 generates and displays numerical values and/or waveforms based on the data received from the sensors for monitoring the one or more physiological variables of the patient. The medical device 100 displays the numerical values and/or waveforms of the one or more physiological variables on the display 104. Also, the medical device 100 generates alarms when the data received from the sensors indicates an alarm condition is detected.

The alarms generated by the medical device 100 can include a visual alarm displayed on the display 104 and an audible alarm emitted by a speaker of the medical device 100. An additional visual alarm can be displayed on an illumination unit 103, which is positioned on the top surface 111 of the housing 102 of the medical device 100. The illumination unit 103 allows for distant viewing of critical alarms. The critical alarms generated on the illumination unit 103 can be viewed across a 360 degree field of view around the medical device 100. In some examples, both a visual alarm is displayed on the display 104 and a second visual alarm is generated on the illumination unit 103 to indicate a critical status of a detected alarm condition.

The illumination unit 103 can include one or more light-emitting diodes (LEDs) that emit light that is visible through a translucent cover. As an illustrative example, the illumination unit 103 can emit light having a color (e.g., yellow or red) based on a severity of the alarm. Additionally, or alternatively, the illumination unit 103 can emit light flashes based on a predetermined pattern associated with the severity of the alarm (e.g., an increased blinking rate to indicate severe conditions and a decreased blinking rate to indicate less severe conditions).

As further shown in the example of the medical device 100 illustrated in FIGS. 1-4, the housing 102 further includes a plurality of vents 116 for dissipating hot air away from internal components of the medical device. The vents 116 are positioned at different areas of the housing 102 such as on the bottom surface 105 (FIG. 4) and the rear surface 107 (FIG. 2).

FIG. 5 schematically illustrates an example of the medical device 100. In this illustrative example, the medical device 100 includes a computing device 120 having at least one processing device 122 and at least one memory device 124 that stores software instructions that, when executed by the at least one processing device 122, cause the at least one processing device 122 to perform the various aspects, functions, and operations described herein. The at least one processing device 122 is communicatively coupled to at least the display 104 and to at least one sensor that captures measurements of a physiological variable.

The at least one processing device 122 is an example of a processing unit such as a central processing unit (CPU). The at least one processing device 122 can include one or more CPUs. In some examples, the at least one processing device 122 includes one or more digital signal processors, field-programmable gate arrays, or other processing circuits.

The at least one memory device 124 is an example of a computer-readable data storage device that operates to store data and instructions for execution by the at least one processing device 122. The at least one memory device 124 includes computer-readable media, which includes any media that can be accessed by the at least one processing device 122.

In the example illustrated in FIG. 5, the at least one memory device 124 stores an improvement mode tool 126 that when executed by the at least one processing device 122 causes the medical device 100 to provide a setup assistant for an improvement alarm mode that can be executed when the medical device 100 is being used to continuously monitor a patient.

The improvement alarm mode when executed on the medical device 100 reduces non-actionable alarms for one or more physiological variables that are monitored by the medical device 100 during a defined time period. When operating under the improvement alarm mode, the medical device 100 generates an alarm only upon further deterioration of the one or more physiological variables. For example, when the one or more physiological variables do not follow a predicted improvement path over a specified time period, the medical device 100 generates an alarm. Since the improvement alarm mode is time-limited, there is a safeguard against the possibility of a user expanding the alarm limits and forgetting to reset them.

The improvement mode tool 126 when executed on the medical device 100 enables a user to initiate and review the new alarm limits set by the improvement alarm mode, as well as to select a specified time period for the improvement alarm mode. The improvement mode tool 126 further enables the user of the medical device 100 to cancel the improvement alarm mode at any time. The improvement mode tool 126 will be described in more detail further below.

The computer-readable media can include computer-readable storage media and computer-readable communication media. The computer-readable storage media includes volatile and nonvolatile, removable and non-removable media implemented in any device that stores information such as computer-readable instructions, data structures, program modules, or other data. The computer-readable storage media can include random access memory, read only memory, electrically erasable programmable read only memory, flash memory, and other memory technology, including any medium that can be used to store information for access by the at least one processing device 122. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

As further shown in FIG. 5, the medical device 100 includes a network interface 128 that facilitates connection to a network 20 that can include any type of wired or wireless connections or any combinations thereof. The network interface 128 can include wired interfaces and/or wireless interfaces. For example, the network interface 128 can wirelessly connect the medical device 100 to the network 20 such as through Wi-Fi. Alternatively, or additionally, the network interface 128 can connect the medical device 100 to the network 20 using wired connections such as through Ethernet or Universal Serial Bus (USB) cables.

The network interface 128 enables the medical device 100 to communicate via the network 20 with an electronic medical record (EMR) system 30. The EMR system 30 maintains the medical history of a plurality of patients by consolidating health information for each patient into a single document called an electronic medical record (EMR). Each EMR can include patient demographic information, medical diagnoses, treatment plans, medications, test results, physiological variable measurements, and immunization history. The EMR system 30 enables sharing of data among healthcare professionals to improve coordination of care, reduce errors, and enhance patient outcomes. The EMR system 30 also supports clinical decision-making by providing real-time access to comprehensive patient health information. The EMR system 30 can include secure data storage and integration with other healthcare technologies, which streamline administrative tasks and improve overall healthcare efficiency.

The medical device 100 further includes a sensor interface 130 that operates to communicate with the various sensors that can be communicatively coupled to the medical device 100 such as the NIBP sensor 106, the thermometer module 108, and the pulse oximeter sensor 112. The sensor interface 130 can include both wired interfaces and wireless interfaces. Examples of wireless interfaces can include Wi-Fi, Bluetooth, and other wireless connections. Examples of wired interfaces can include the ports of the medical device 100 (see FIG. 4) that allow connectorized ends of USB or Ethernet cables to plug into the sensor interface 130.

As further shown in FIG. 5, a speaker 132 serves as an integral component of the medical device 100 for providing clear auditory feedback for users and healthcare professionals. The speaker 132 can be used to deliver audible alarms, alerts, notifications, and/or instructions. The speaker 132 ensures that vital information is communicated effectively. Designed for precision and clarity, the speaker 132 helps enhance patient safety by making sure critical data is heard, even in busy or noisy environments which are typical in medical settings.

As shown in FIG. 5, the medical device 100 includes the display 104 which operates to display user interfaces 134. The display 104 can include a touch sensitive screen (e.g., a touchscreen) that receives tactile inputs from the touch of a finger or stylus on its surface. In such examples, the display 104 can include a grid of capacitive or resistive sensors to register locations of touch. In some examples, the display 104 includes a capacitive touchscreen that is coated with a conductive material. When a finger or stylus touches the capacitive touchscreen, the local electrostatic field produced by the conductive material of display 104 changes, allowing the medical device 100 to determine a point of contact. Alternatively, the display 104 can include a resistive touchscreen that registers pressure applied to the screen by detecting change in resistance when layers of the screen come into contact. The tactile inputs, whether capacitive or resistive, are processed by the computing device 120 to identify actions such as selecting or dragging visual objects that are displayed on the user interfaces 134.

When the vitals of a patient who is being monitored by the medical device 100 are outside default alarm limits, but are expected to improve, users can let the medical device 100 produce non-actionable alarms. However, this will increase non-actionable alarms which can overwhelm the users leading to alarm fatigue which can cause the users to begin to consciously and unconsciously ignore the alarms generated by the medical device 100, which can cause them to miss patient deterioration. Additionally, these non-actionable alarms will add unnecessary stress and noise to the patient environment. As an alternative, the users of the medical device 100 can manually update the alarm limits to align with the current status or condition of the patient. However, the users can update the alarm limits inappropriately (i.e., setting an upper alarm limit too high or a lower alarm limit too low). Further, the users may forget to reset the alarm limits once the treatment of the patient is completed which can be dangerous because the manually adjusted alarm limits would not detect patient deterioration appropriately.

The improvement alarm mode can be executed on the medical device 100 when one or more physiological variables are abnormal but are expected to improve or remain stable over a specified time period such as, for example, due to treatment, intervention, or recovery. However, the improvement alarm mode is intended for use by advanced users because the configuration of the improvement alarm mode can be troublesome for inexperienced users who are not aware of how the patient data is used to set new alarm limits for monitoring the one or more physiological variables, or the time period that is used for automatically adjusting the alarm limits towards default alarm limits as the one or more physiological variables are expected to improve.

The improvement mode tool 126 is directed to solving the aforementioned technical problems by assisting the users of the medical device 100 with the setup, initiation, and utilization of the improvement alarm mode. The improvement mode tool 126 provides a quick guided setup for the improvement alarm mode that tracks progress of one or more physiological variables against predicted improvement paths. The improvement mode tool 126 allows users of the medical device 100 to make informed decisions about next steps such as whether to extend the improvement alarm mode, or to cancel the improvement alarm mode, or to try a different treatment when the one or more physiological variables of the patient do not improve.

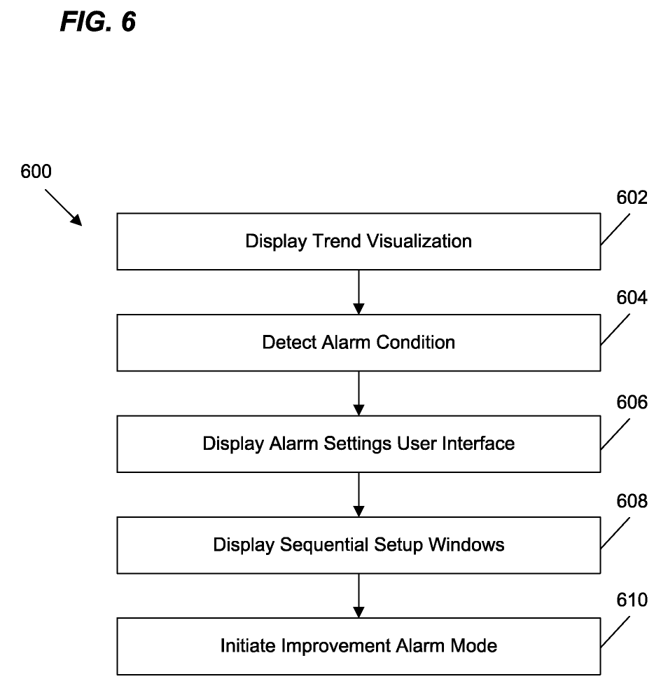

FIG. 6 schematically illustrates an example of a method 600 of managing alarms on the medical device 100. The method 600 can be performed by the improvement mode tool 126 executed on the medical device 100. The method 600 can be executed on the medical device 100 to configure and setup the improvement alarm mode for monitoring one or more of blood oxygen saturation (SpO2), pulse rate, respiration rate, and non-invasive blood pressure (NIBP).

The method 600 includes an operation 602 of displaying on the display 104 a trend visualization showing measured values of a physiological variable relative to default alarm limits that include a default upper alarm limit and a default lower alarm limit. The measured values can be based on data from one or more sensors communicatively coupled to the medical device 100 such as the NIBP sensor 106, the thermometer module 108, and the pulse oximeter sensor 112.

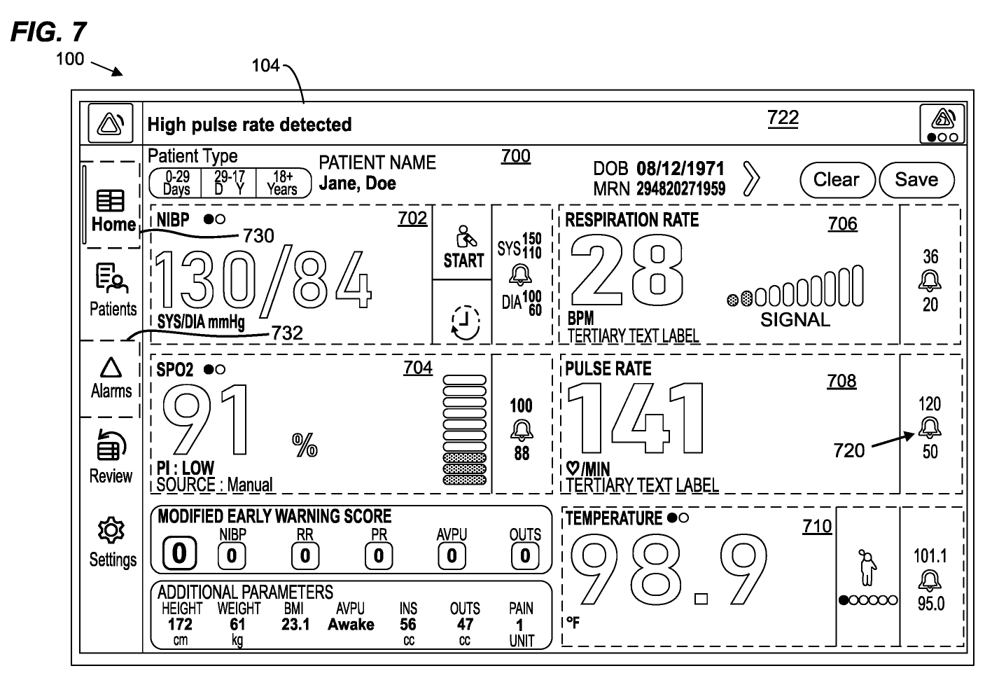

FIG. 7 illustrates an example of a home screen user interface 700 that can be displayed on the display 104 of the medical device 100. The home screen user interface 700 is displayed in response to receiving a tactile input on a home tab 730. In this illustrative example, the home screen user interface 700 includes a non-invasive blood pressure (NIBP) display area 702 that displays systolic and diastolic blood pressure measurements based on the data received from the NIBP sensor 106. The home screen user interface 700 further includes a blood oxygen saturation (SpO2) display area 704 that displays SpO2 measurements, a respiration rate display area 706 that displays respiration rate measurements, and a pulse rate display area 708 that displays pulse rate measurements based on the data received from the pulse oximeter sensor 112. The home screen user interface 700 further includes a temperature display area 710 that displays temperature measurements based on data from the thermometer module 108.

Referring back to FIG. 6, the method 600 includes an operation 604 of detecting an alarm condition. The alarm condition is detected when the measured values of the physiological variable exceed the default upper alarm limit or when the measured values of the physiological variable are less than the default lower alarm limit. Operation 604 can include comparing the measurement values of the physiological variable to default upper and lower alarm limits.

In the example shown in FIG. 7, an alarm condition is detected based on the pulse rate measurements displayed in the pulse rate display area 708. The pulse rate display area 708 displays a pulse rate measurement of 141 beats per minute (bpm) which exceeds an upper alarm limit of 120 bpm. In this example, a lower alarm limit of 50 bpm is set for the pulse rate.

As shown in FIG. 7, when the alarm condition is detected by the medical device 100, a border of the display area with the detected alarm condition, in this case the pulse rate display area 708, is illuminated (e.g., in the color red) to distinguish the display area from the other display areas of the home screen user interface 700. Also, an alarm icon 720 is illuminated (e.g., in the color red) inside the display area with the alarm condition. Further, an alarm ribbon 722 is displayed across the home screen user interface 700 such as across a top portion of the interface. The alarm ribbon 722 identifies the alarm condition (e.g., “High pulse rate detected”).

Referring back to FIG. 6, the method 600 includes an operation 606 of displaying an alarm settings user interface in response to receiving a tactile input on the display 104 of the medical device 100. The alarm settings user interface allows a user of the medical device 100 to adjust settings for alarms that are used for monitoring the one or more physiological variables.

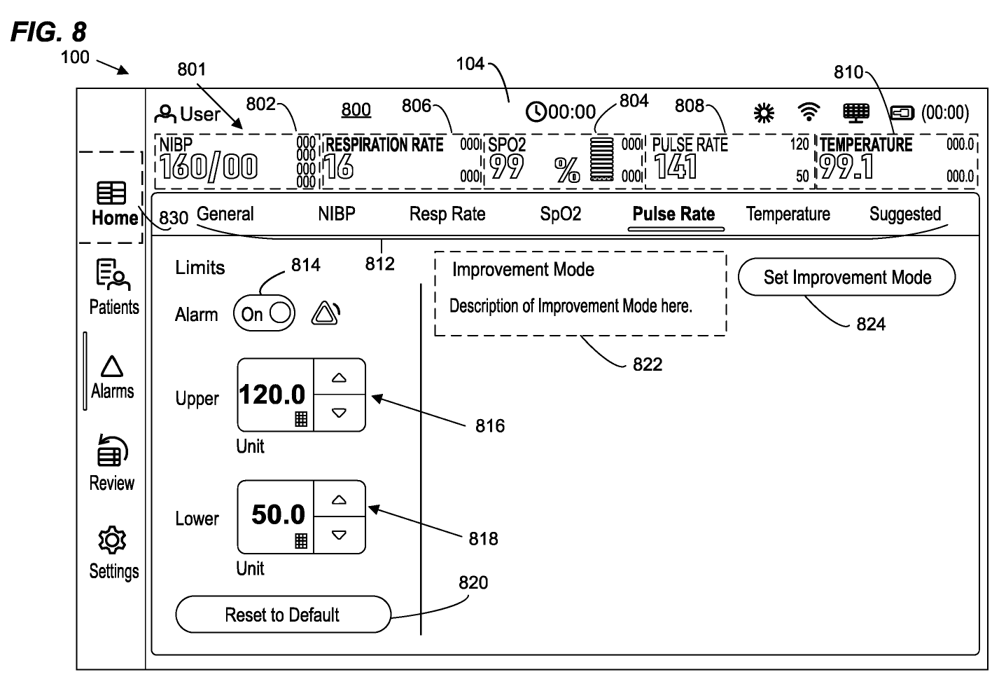

FIG. 8 illustrates an example of an alarm settings user interface 800 displayed on the display 104 of the medical device 100. The alarm settings user interface 800 is displayed in response to receiving a tactile input on an alarm tab 732 in the home screen user interface 700 of FIG. 7. Alternatively, the alarm settings user interface 800 is displayed in response to receiving a tactile input on the alarm icon 720 displayed inside the display area of the physiological variable that is causing the alarm condition to be triggered (e.g., the pulse rate display area 708).

The alarm settings user interface 800 includes a monitoring area 801 that includes a NIBP display area 802 that displays systolic and diastolic blood pressure measurements; a SpO2 display area 804 that displays SpO2 measurements; a respiration rate display area 806 that respiration rate measurements; a pulse rate display area 808 that displays pulse rate measurements; and a temperature display area 810 that displays temperature measurements.

The alarm settings user interface 800 further includes a plurality of tabs 812 for adjusting the alarm settings for each physiological variable monitored by the medical device. The plurality of tabs 812 includes a tab for general alarm settings, a tab for adjusting alarms for monitoring NIBP measurements, a tab for adjusting alarms for monitoring respiration rate measurements, a tab for adjusting alarms for monitoring SpO2 measurements, a tab for adjusting alarms for monitoring pulse rate measurements, a tab for adjusting alarms for monitoring temperature measurements, and a tab for implementing suggested alarms for monitoring measurements of one or more of the physiological variables. In the example illustrated in FIG. 8, the tab for adjusting alarms for monitoring pulse rate measurements is shown as selected.

The alarm settings user interface 800 can include a toggle switch 814 that allows selection between turning on and off the alarms for the physiological variable. In the example shown in FIG. 8, the toggle switch 814 is switched on such that alarming on the medical device 100 for the pulse rate measurements is turned on. The alarm settings user interface 800 can also include an upper alarm limit adjuster 816 that includes up and down arrows to adjust a numeral value for the upper alarm limit for monitoring the pulse rate measurements, and a lower alarm limit adjuster 818 that includes up and down arrows to adjust a numeral value for the lower alarm limit for monitoring the pulse rate measurements. In some examples, access to the toggle switch 814, the upper alarm limit adjuster 816, and the lower alarm limit adjuster 818 is restricted only for users who possess credentials that provide a higher level of access or permission. The alarm settings user interface 800 can also include a reset icon 820 that when selected causes the upper and lower alarm limits to return to their default values.

As further shown in FIG. 8, the alarm settings user interface 800 includes an improvement mode message area 822 that describes the improvement alarm mode such as what it does, how it works, and the potential advantages of executing it on the medical device 100. The alarm settings user interface 800 further includes a set improvement mode icon 824 that when select launches the improvement mode tool 126, as discussed further below.

Referring back to FIG. 6, the method 600 includes an operation 608 of displaying sequential setup windows that guide clinical workflow for initiating the improvement alarm mode on the medical device 100 for monitoring one or more physiological variables. The improvement mode tool 126 controls the display of the sequential setup windows on the display 104 for configuring the improvement alarm mode for execution on the medical device 100.

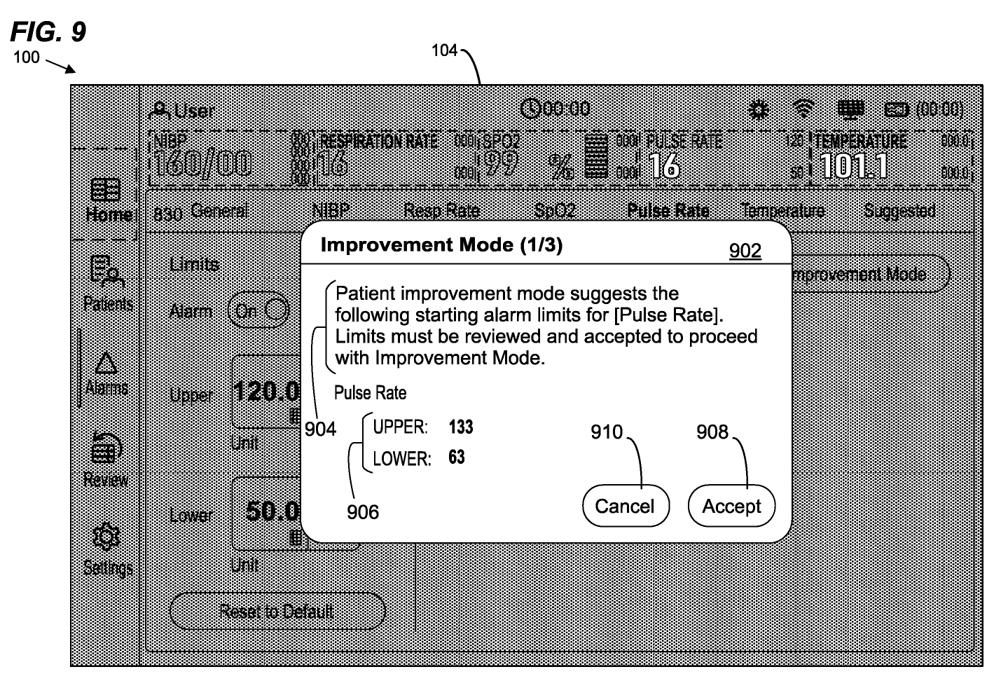

FIG. 9 illustrates an example of a first setup window 902 that can be displayed on the display 104 of the medical device 100 as a result of selecting the set improvement mode icon 824 in the alarm settings user interface 800 of FIG. 8. The first setup window 902 includes a message 904 that describes the need for a user to review and accept starting alarm limits suggested by the improvement alarm mode (e.g., “Patient improvement mode suggests the following starting alarm limits for [Pulse Rate]. Limits must be reviewed and accepted to proceed with improvement mode.”). The first setup window 902 displays the starting alarm limits 906 that are suggested by improvement alarm mode (e.g., a starting upper limit of 133 beats per minute (bpm) which is 13 bpm higher than the default upper limit of 120 bpm and a starting lower limit of 63 bpm which is 13 bpm higher than the default lower limit of 50 bpm). The starting alarm limits 906 are calculated by the improvement mode tool 126 based on recent measurements of the physiological variable that is causing the alarm condition (i.e., pulse rate).

The first setup window 902 further includes an accept icon 908 that when selected causes the improvement mode tool 126 to display a second setup window. The first setup window 902 includes a cancel icon 910 that when selected causes the improvement mode tool 126 to close the first setup window 902 and return to displaying the alarm settings user interface 800 shown in FIG. 8 without initiating the improvement alarm mode on the medical device 100.

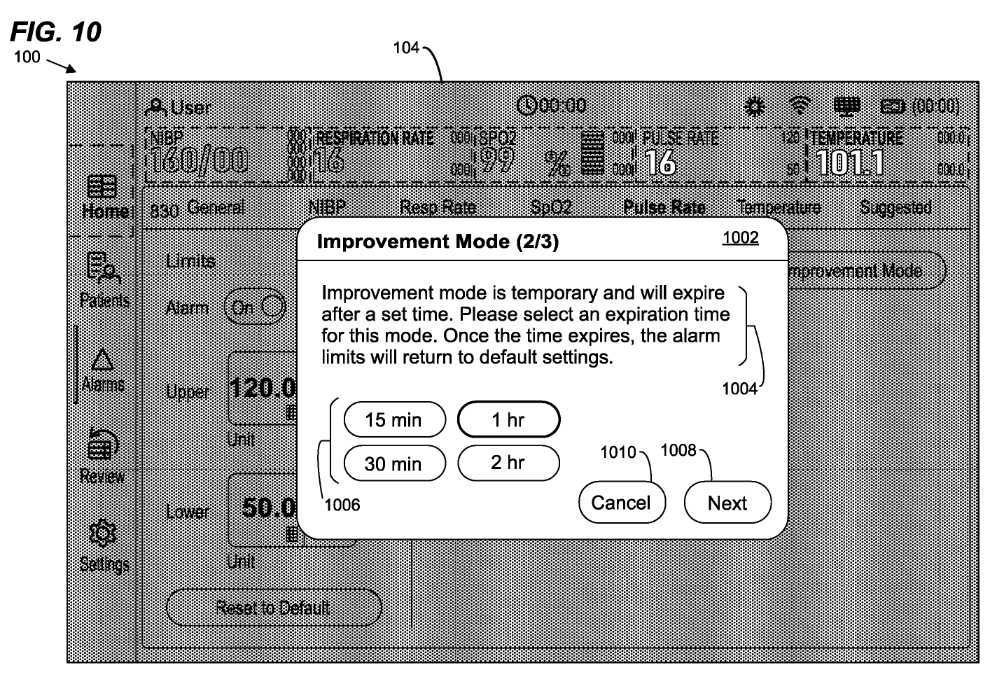

FIG. 10 illustrates an example of a second setup window 1002 that can be displayed on the display 104 of the medical device 100 as a result of selecting the accept icon 908 in the first setup window 902 shown in FIG. 9. The second setup window 1002 includes a message 1004 that states that the improvement alarm mode is temporary, and that the user needs to select an expiration time for the improvement alarm mode (e.g., “Improvement mode is temporary and will expire after a set time. Please select an expiration time for this mode. Once the time expires, the alarm limits will return to default settings.”). The second setup window 1002 displays one or more expiration times 1006 for selection by the user (e.g., 15 minutes, 30 minutes, 1 hour, and 2 hours). In the example shown in FIG. 10, an expiration time of 1 hour is shown as selected.

The second setup window 1002 further includes a next icon 1008 that when selected causes the improvement mode tool 126 to display a third setup window. The second setup window 1002 includes a cancel icon 1010 that when selected causes closure of the second setup window 1002 and return to displaying the alarm settings user interface 800 shown in FIG. 8 without initiating the improvement alarm mode on the medical device 100.

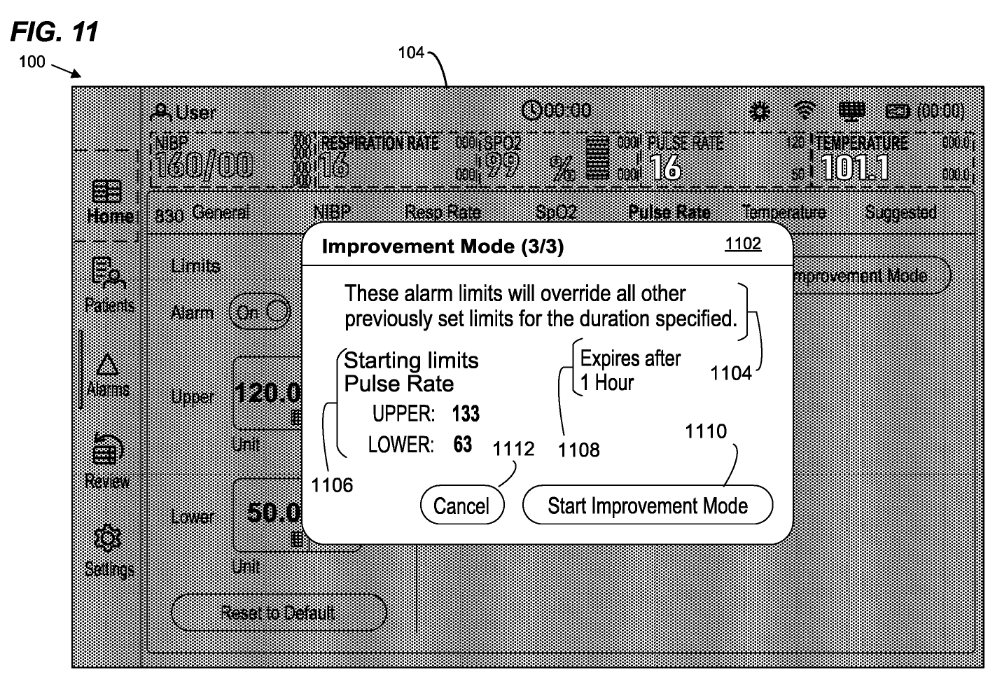

FIG. 11 illustrates an example of a third setup window 1102 that can be displayed on the display 104 of the medical device 100 as a result of selecting the next icon 1008 in the second setup window 1002 shown in FIG. 10. The third setup window 1102 includes a message 1104 that states the starting alarm limits accepted in the first setup window 902 will override all other previously set alarm limits for the duration selected in the second setup window 1002 (e.g., “These alarm limits will override all other previously set limits for the duration specified.”). The third setup window 1102 displays a summary 1106 of the starting alarm limits suggested by the improvement mode tool 126 and accepted in the first setup window 902. The third setup window 1102 further displays a summary 1108 of the duration of the improvement alarm mode based on the expiration time selected in the second setup window 1002.

The third setup window 1102 further includes a start icon 1110 that when selected initiates the improvement alarm mode on the medical device 100. The third setup window 1102 includes a cancel icon 1112 that when selected causes closure of the third setup window 1102 and return to displaying the alarm settings user interface 800 shown in FIG. 8 without initiating the improvement alarm mode on the medical device 100. Accordingly, the improvement alarm mode cannot be initiated on the medical device 100 unless a user goes through the sequential setup windows (e.g., first, second, and third setup windows 902-1102) for configuring the improvement alarm mode for execution on the medical device 100. This can ensure that the user understands the parameters of the improvement alarm mode such that it is not initiated in error.

Referring back to FIG. 6, the method 600 includes an operation 610 of initiating the improvement alarm mode on the medical device 100. Operation 610 occurs in response to selection of the start icon 1110 in the third setup window 1102 of FIG. 11. Once initiated, the improvement alarm mode terminates when the automatically adjusting alarm limits reach the default alarm limits, the duration selected for the improvement alarm mode expires, or an input is received via the display 104 to cancel the improvement alarm mode as discussed below. Upon termination of the improvement alarm mode, the default alarm limits return for monitoring the measurements of the physiological variable.

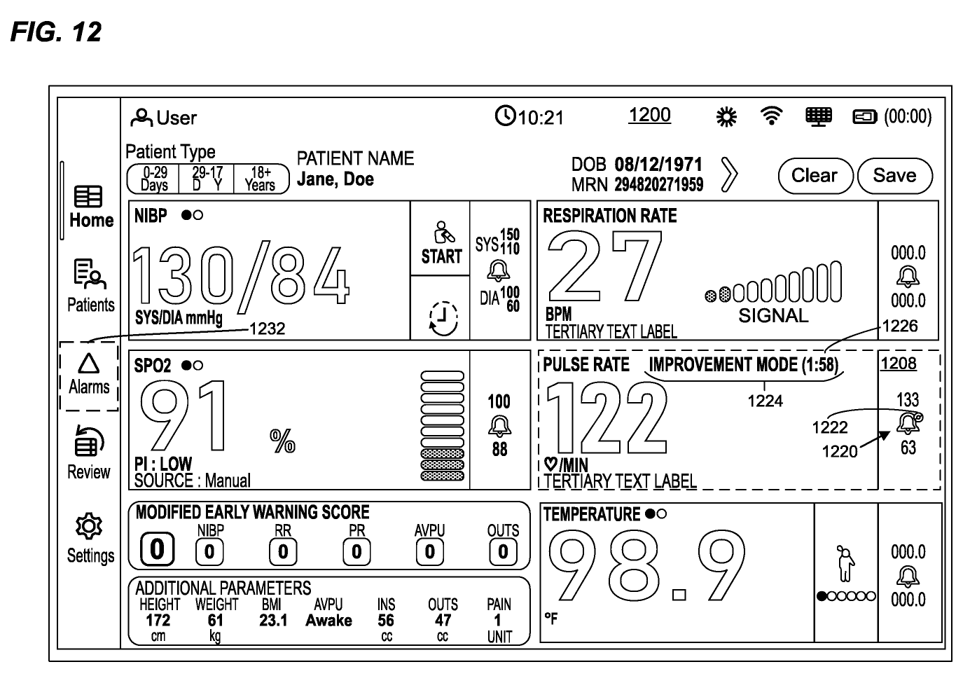

FIG. 12 illustrates another example of a home screen user interface 1200 that can be displayed on the display 104 of the medical device 100 following initiation of the improvement alarm mode in accordance with operation 610 of the method 600. The home screen user interface 1200 is displayed in response to receiving a tactile input on a home tab 830 of the alarm settings user interface 800 of FIG. 8 after the improvement alarm mode is initiated by going through the sequential setup windows shown in FIGS. 9-11. In the example shown in FIG. 12, the pulse rate display area 1208 is updated to show that the improvement alarm mode is being used to monitor the pulse rate. For example, the pulse rate display area 1208 is updated to display a message 1224 that indicates the improvement alarm mode is being used to monitor the pulse rate. The message 1224 can include a countdown timer 1226 showing the time remaining before the improvement alarm mode expires (e.g., “1:58” to indicate 1 hour 58 minutes remaining).

The alarm icon 1220 can be illuminated to have a color (e.g., blue) that is different than the color that illuminates the alarm icon 720 in FIG. 7 when the alarm condition is initially detected. Also, the current alarm limits applied by the improvement alarm mode for monitoring the physiological variable are displayed inside the pulse rate display area 1208. For example, a current upper alarm limit can be displayed above the alarm icon 1220, and a current lower alarm limit can be displayed below the alarm icon 1220. Further, a symbol 1222 can be displayed next to the current alarm limits to indicate that they are automatically updating.

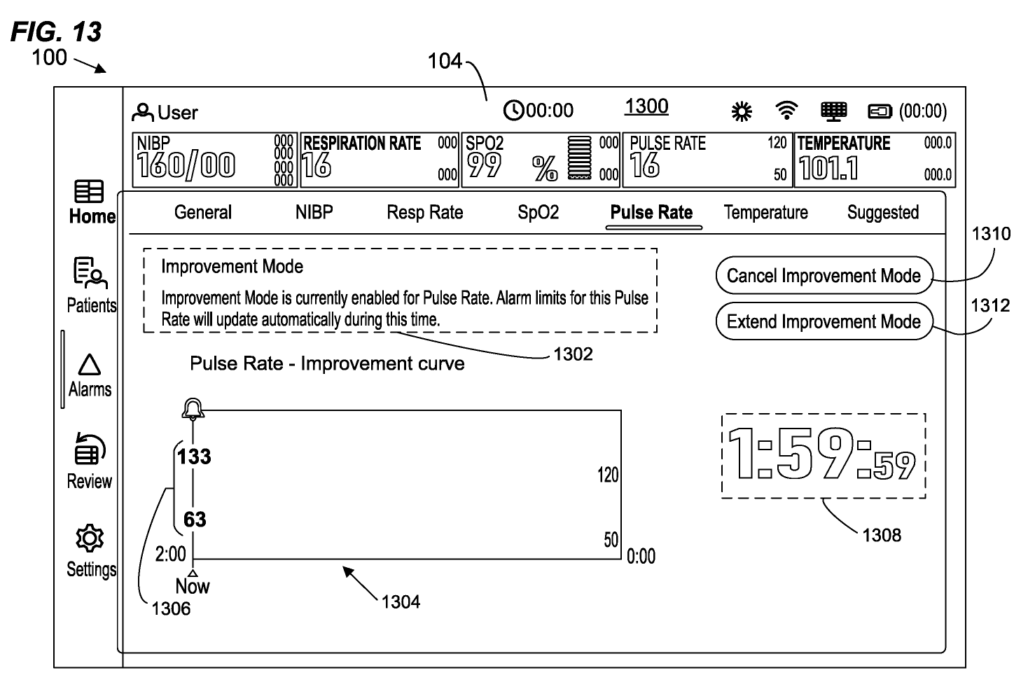

FIG. 13 illustrates another example of an alarm settings user interface 1300 displayed on the display 104 of the medical device 100. The alarm settings user interface 1300 is displayed in response to receiving a tactile input on an alarm tab 1232 in the home screen user interface 1200. The alarm settings user interface 1300 is updated to show the improvement alarm mode is being used to monitor the pulse rate. For example, the alarm settings user interface 1300 has a message area 1302 that displays a message to alert users that the improvement alarm mode is currently enabled for pulse rate (e.g., “ Improvement mode is currently enabled for pulse rate. Alarm limits for pulse rate will update automatically during this time.”).

The alarm settings user interface 1300 includes a visualization 1304 for showing measured values of pulse rate relative to automatically adjusting alarm limits that begin with starting alarm limits 1306 and gradually increase or decrease to follow a predicted improvement curve. In FIG. 13, the improvement alarm mode has just begun such that there are no measured values of pulse rate for comparison with the predicted improvement curve in the visualization 1304. The fact that the improvement alarm mode has just begun is evidenced by a countdown timer 1308 which shows that there is 1 hour, 59 minutes, and 59 seconds remaining for the improvement alarm mode which is configured to have an expiration time of two hours.

Instead, the visualization 1304 only shows starting alarm limits 1306 suggested by the improvement mode tool 126. In this example, the starting alarm limits 1306 include a starting upper limit of 133 bpm which is 13 bpm higher than the default upper limit of 120 bpm and a starting lower limit of 63 bpm which is 13 bpm higher than the default lower limit of 50 bpm.

The alarm settings user interface 1300 includes a cancel improvement mode icon 1310 that when selected cancels the improvement alarm mode such that the default alarm limits return for monitoring the physiological variable (e.g., pulse rate). The alarm settings user interface 1300 also includes an extend improvement mode icon 1312 that when selected extends the improvement alarm mode for a preset time period. Accordingly, a user of the medical device 100 can cancel or extend the improvement alarm mode at any time including before expiration of its duration as identified in the countdown timer 1308.

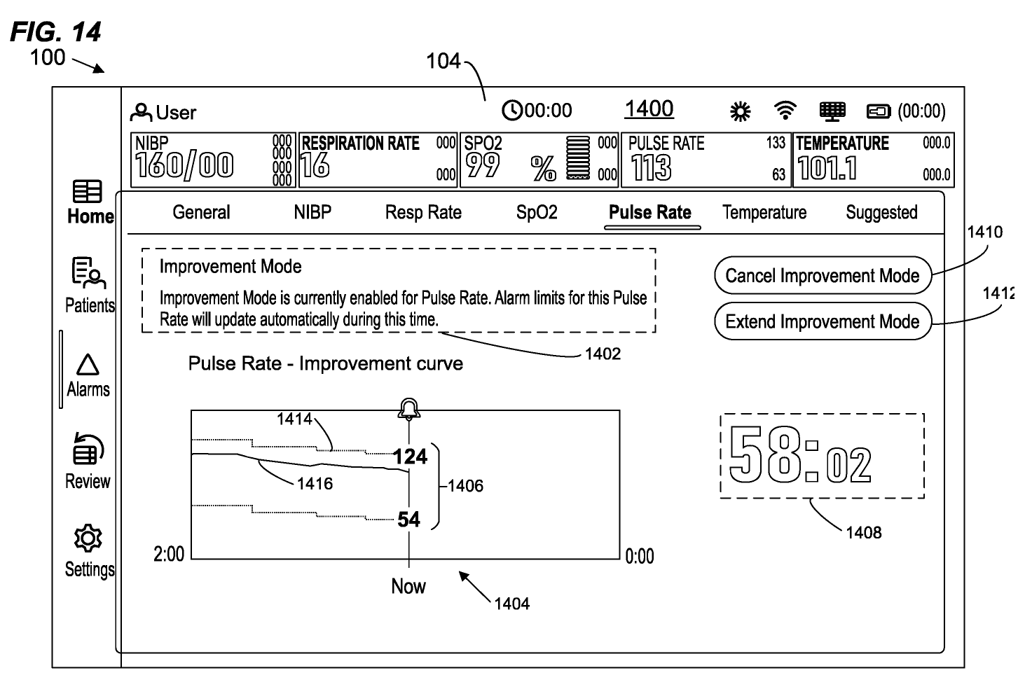

FIG. 14 illustrates another example of an alarm settings user interface 1400 displayed on the display 104 of the medical device 100 after the improvement alarm mode has been initiated on the medical device 100. In this example, the improvement alarm mode has progressed in comparison to the alarm settings user interface 1300 of FIG. 13 as evidenced by the countdown timer 1408 indicating that there are 58 minutes and 2 seconds remaining before expiration of the improvement alarm mode. Accordingly, the visualization 1404 is updated to show a trend of pulse rate measurements 1416 relative to automatically adjusting alarm limits 1406 that began with the starting alarm limits shown in FIG. 13 and that have gradually decreased to follow a predicted improvement curve 1414 towards the default alarm limits.

In the example of FIG. 14, the trend of pulse rate measurements 1416 is improving as expected such that it is following the predicted improvement curve 1414 in a downward trajectory towards the default alarm limits. The automatically adjusting alarm limits 1406 have a current upper limit of 124 bpm (i.e., 4 bpm higher than the default upper limit of 120 bpm) and a current lower limit of 54 bpm (i.e., 4 bpm higher than the default lower limit of 50 bpm).

Like the example shown in FIG. 13, the alarm settings user interface 1400 similarly includes the message area 1402 that displays the message to alert users that the improvement alarm mode is currently enabled for pulse rate. The alarm settings user interface 1400 also includes the cancel improvement mode icon 1410 and the extend improvement mode icon 1412 to cancel or extend the improvement alarm mode at any time prior its expiration.

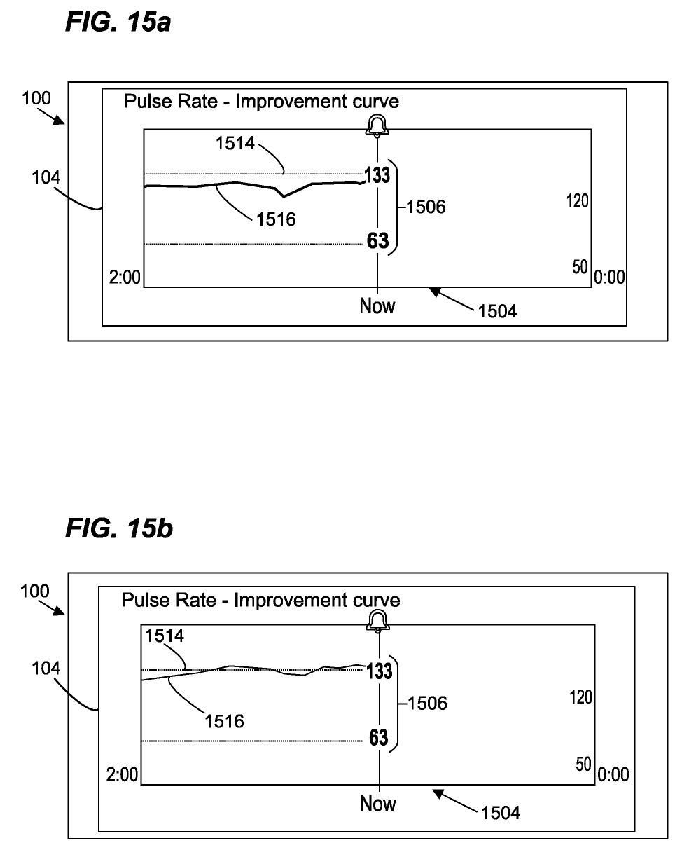

FIG. 15a illustrates another example of a visualization 1504a that can be displayed in the alarm settings user interface 1400 of FIG. 14. In this illustrative example, the trend of pulse rate measurements 1516 is stable such that it is not improving or deteriorating. In this illustrative example, the automatically adjusting alarm limits 1506 have a current upper alarm limit of 133 bpm which is the same as the starting upper alarm limit in FIG. 13 and a current lower alarm limit of 63 bpm which is the same as the starting lower alarm limit in FIG. 13.

FIG. 15b illustrates another example of a visualization 1504b that can be displayed in the alarm settings user interface 1400 of FIG. 14. In this example, the trend of pulse rate measurements 1516 is deteriorating because it is increasing instead of decreasing toward the default alarm limits. The improvement alarm mode causes the medical device 100 to generate an alarm because the trend of pulse rate measurements 1516 is exceeding a current upper alarm limit of 133 bpm which is the same as the starting upper alarm limit in FIG. 13. Thus, alarm functionality is maintained for detecting deterioration during the improvement alarm mode.

The trend of the pulse rate measurements 1516 can use different colors or dash types to show when the measurements exceed the upper alarm limit and when they do not. For example, the trend of pulse rate measurements 1516 has a color (e.g., blue) when below the upper alarm limit and the trend of pulse rate measurements has another color (e.g., red) when above the upper alarm limit. Accordingly, upon activation of the improvement alarm mode, visual indicators are provided to show whether measured values of a physiological variable are following, deviating from, or opposing the automatically adjusting alarm limits. Further, the improvement alarm mode generates an alarm when the measured values of the physiological variable indicate patient deterioration relative to the automatically adjusting alarm limits.

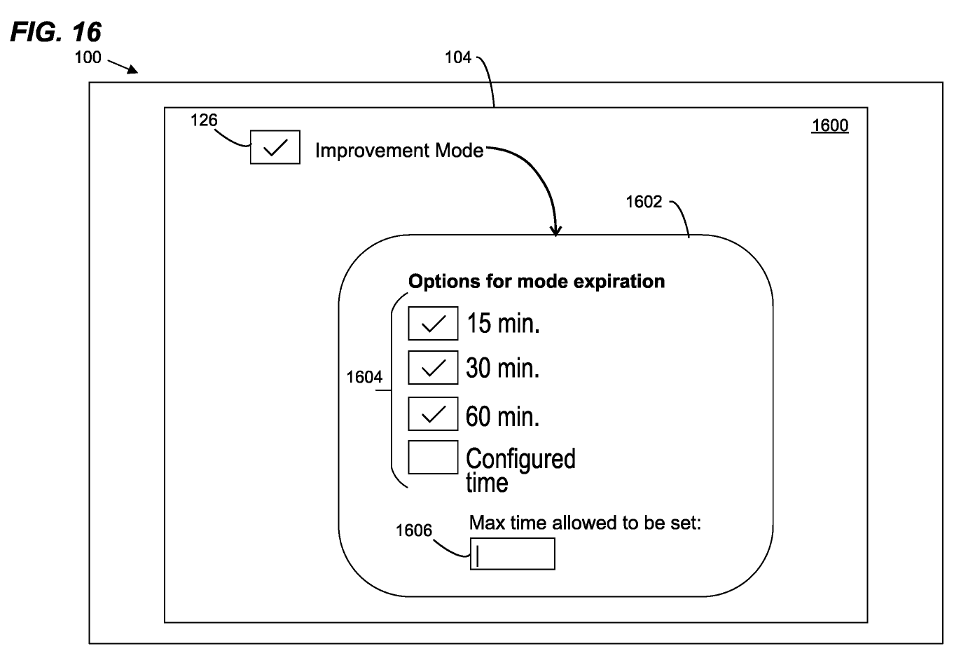

FIG. 16 illustrates an example of a configuration user interface 1600 that can be displayed on the display 104 of the medical device 100 to configure the improvement mode tool 126. In this example, the configuration user interface 1600 displays an improvement mode expiration window 1602 that includes a set of inputs 1604 for selection of one or more expiration times or durations for the improvement alarm mode. The set of inputs 1604 includes graphical user interface elements (e.g., empty squares) that when selected cause a marker such as a checkmark to appear inside the element to indicate the selection. Clicking on an element twice deselects the element such that the mark disappears. In the illustrated example, the set of inputs 1604 includes elements for selecting one or more of 15 minutes, 30 minutes, 60 minutes, or a configurable time, with the 15 minutes, 30 minutes, 60 minutes each being shown as selected. This causes the second setup window 1002 shown in FIG. 10 to display expiration times 1006 that include 15 minutes, 30 minutes, or 60 minutes for selection by a user of the medical device 100. Thus, the expiration times 1006 displayed in the second setup window 1002 are preset.

In addition to the preset times that are available for selection in the set of inputs 1604, an authorized user such as an administrator has the option to offer other users the ability to configure their own improvement mode expiration time by selecting the element for the configurable time in the set of inputs 1604. This allows the other users to enter a configurable expiration time instead of selecting one of the preset expiration times. This can be advantageous for experienced users who would like to enter a configurable expiration time for the improvement alarm mode based on context including the care area, the patient, and the environment around the patient. When the element for selecting the configurable expiration time is selected in the set of inputs 1604, a maximum time must be set using entry field 1606 such that the configurable expiration time cannot exceed the maximum time to ensure patient safety.

In some examples, configuration user interface 1600 can be used to configure the availability of the improvement alarm mode based on at least one of healthcare area and patient type. For example, the improvement alarm mode can be made available for execution on the medical device 100 for certain areas of a healthcare facility (e.g., med-surg unit) but not other areas (e.g., intensive care unit (ICU)). Similarly, the improvement alarm mode can be made available for execution on the medical device 100 for certain types of patients (e.g., adult patients) but not other types of patients (e.g., neonatal patients).

The medical device 100 provides specific technical improvements over contemporary monitoring systems by implementing the improvement mode tool 126 for configuring the improvement alarm mode to reduce non-actionable alarms while maintaining critical patient safety monitoring. The improvement mode tool 126 provides a concrete technical solution to the problem of configuring the improvement alarm mode to mitigate alarm fatigue in healthcare environments by automatically adjusting alarm limits based on predicted improvement curves.

The improvement mode tool 126 implements a specific technological solution through sequential setup windows that guide clinical workflow, ensuring proper configuration of the improvement alarm mode through a structured process requiring active user validation. This represents a practical application that goes beyond mere data gathering or display, as it provides an interactive technical framework for implementing sophisticated alarm management system.

The technology provides tangible improvements to the functioning of medical device 100 by reducing non-actionable alarms that can overwhelm users and lead to alarm fatigue, while maintaining critical alarm functionality for detecting patient deterioration. The medical device 100 includes specific technical features like automatically adjusting alarm limits that follow predicted improvement curves, visual indicators showing measured values relative to these dynamic limits, and automatic termination of the improvement mode to ensure patient safety.

The medical device 100 integrates various technical components including touchscreen displays, sensors, processing devices, and memory in a specific arrangement that enables new functionality for medical device alarm management. The technology provides specific improvements to the technical field of medical device alarm management through its structured implementation of the improvement alarm mode, including guided setup through sequential windows, automated adjustment of alarm limits along predicted improvement curves, and maintenance of critical alarm functionality. These features demonstrate that the medical device provides concrete technical solutions to technological problems in alarm management. The improvement alarm mode works in conjunction with these features to provide a complete solution for reducing non-actionable alarms while maintaining patient safety.

The various embodiments described above are provided by way of illustration only and should not be construed to be limiting in any way. Various modifications can be made to the embodiments described above without departing from the true spirit and scope of the disclosure.

What is claimed is:

1. A medical device comprising:
at least one processing device communicatively coupled to a display and to at least one sensor that captures measurements of a physiological variable; and
a memory device storing instructions which, when executed by the at least one processing device, cause the at least one processing device to:
display on the display a trend visualization showing measured values of the physiological variable relative to default alarm limits that include a default upper alarm limit and a default lower alarm limit;
detect an alarm condition when the measured values exceed the default upper alarm limit or when the measured values are less than the default lower alarm limit;
display setup windows that guide clinical workflow for initiating an improvement alarm mode, the setup windows including:
a first window showing suggested starting alarm limits based on recent measurements of the physiological variable, the starting alarm limits including at least one of a starting upper alarm limit differing from the default upper alarm limit and a starting lower alarm limit differing from the default lower alarm limit;
a second window for selecting an expiration time for the improvement alarm mode; and
a third window summarizing the suggested starting alarm limits and the expiration time for the improvement alarm mode before activation; and
upon activation of the improvement alarm mode, display a visualization showing the measured values relative to automatically adjusting alarm limits that begin with the starting alarm limits and gradually increase or decrease to follow a predicted improvement curve.

2. The medical device of claim 1, wherein the memory device stores additional instructions which, when executed by the at least one processing device, further cause the at least one processing device to:
display a configuration interface enabling:
selection of available improvement alarm mode expiration times; and
selection of maximum duration for the improvement alarm mode.

3. The medical device of claim 1, wherein the memory device stores additional instructions which, when executed by the at least one processing device, further cause the at least one processing device to:
cause the display to provide:
a countdown timer for a remaining duration for the improvement alarm mode;
visual indicators of whether the measured values are following, deviating from, or opposing the automatically adjusting alarm limits; and
controls for extending or canceling the improvement alarm mode.

4. The medical device of claim 1, wherein the memory device stores additional instructions which, when executed by the at least one processing device, further cause the at least one processing device to:
maintain alarm functionality for detecting deterioration during the improvement alarm mode by generating an alarm when the measured values of the physiological variable indicate patient deterioration relative to the automatically adjusting alarm limits; and
return to the default alarm limits upon expiration of the improvement alarm mode.

5. A method of managing alarms on a medical device, the method comprising:
displaying a trend visualization showing values of a physiological variable relative to default alarm limits including a default upper alarm limit and a default lower alarm limit;
detecting an alarm condition when the values exceed the default upper alarm limit or when the values are less than the default lower alarm limit;
displaying setup windows that guide clinical workflow for initiating an improvement alarm mode, the setup windows including:
a first window showing suggested starting alarm limits based on recent measurements of the physiological variable, the starting alarm limits including at least one of a starting upper alarm limit differing from the default upper alarm limit and a starting lower alarm limit differing from the default lower alarm limit;
a second window for selecting an expiration time for the improvement alarm mode; and
a third window summarizing the suggested starting alarm limits and the expiration time selected for the improvement alarm mode before activation; and
upon activation of the improvement alarm mode, displaying a visualization showing the values of the physiological variable relative to automatically adjusting alarm limits that begin with the starting alarm limits and gradually increase or decrease to follow a predicted improvement curve.

6. The method of claim 5, further comprising:
terminating the improvement alarm mode when the automatically adjusting alarm limits reach the default alarm limits, a duration of the improvement alarm mode expires, or an input is received to cancel the improvement alarm mode.

7. The method of claim 6, further comprising:
applying the default alarm limits to the values of the physiological variable when the improvement alarm mode terminates.

8. The method of claim 5, further comprising:
updating a home screen to show the improvement alarm mode is being executed on the medical device for monitoring the physiological variable, wherein the home screen displays measurements for a plurality of physiological variables.

9. The method of claim 5, further comprising:
updating a user interface to show a trend of the values of the physiological variable relative to the automatically adjusting alarm limits, a countdown timer showing an amount of time remaining before expiration of the improvement alarm mode, a first option to cancel the improvement alarm mode, and a second option to extend the improvement alarm mode.

10. The method of claim 9, further comprising:
upon selection of the second option to extend the improvement alarm mode, extending the improvement alarm mode for a preset time period.

11. The method of claim 5, further comprising:
generating an alarm when the values of the physiological variable are higher than an upper alarm limit automatically adjusted by the improvement alarm mode or when the values of the physiological variable are lower than a lower alarm limit automatically adjusted by the improvement alarm mode.

12. The method of claim 5, wherein the physiological variable is non-invasive blood pressure, blood oxygen saturation (SpO2), respiration rate, pulse rate, or temperature.

13. A medical device comprising:
at least one processing device communicatively coupled to a display and to at least one sensor that captures measurements of a physiological variable; and
a memory device storing instructions which, when executed by the at least one processing device, cause the at least one processing device to:
generate an alarm when the measurements of the physiological variable are higher than a default upper alarm limit or when the measurements of the physiological variable are lower than a default lower alarm limit;
display a user interface having an input to configure an improvement alarm mode;
upon selection of the input to configure the improvement alarm mode, display a first window having a starting upper alarm limit that differs from the default upper alarm limit and a starting lower alarm limit that differs from the default lower alarm limit;
upon acceptance of the starting upper and lower alarm limits, display a second window displaying one or more expiration times for the improvement alarm mode;
upon selection of an expiration time for the improvement alarm mode, display a third window summarizing the starting upper and lower alarm limits accepted in the first window and the expiration time selected in the second window, the third window including an input to initiate the improvement alarm mode; and
upon selection of the input to initiate the improvement alarm mode, execute the improvement alarm mode for the physiological variable, wherein the improvement alarm mode automatically adjusts the starting upper and lower alarm limits toward the default upper and lower alarm limits.

14. The medical device of claim 13, wherein the improvement alarm mode terminates when the automatically adjusting upper and lower alarm limits reach the default upper and lower alarm limits, when the expiration time is reached, or when an input is selected to cancel the improvement alarm mode.

15. The medical device of claim 14, wherein when the improvement alarm mode terminates, the default upper and lower alarm limits are applied for monitoring the measurements of the physiological variable.

16. The medical device of claim 13, wherein the memory device stores additional instructions which, when executed by the at least one processing device, further cause the at least one processing device to:
update a home screen displayed on the display to show the improvement alarm mode is being executed on the medical device for the physiological variable, wherein the home screen displays measurements for a plurality of physiological variables.

17. The medical device of claim 13, wherein the memory device stores additional instructions which, when executed by the at least one processing device, further cause the at least one processing device to:
update the user interface to show a trend of the measurements of the physiological variable relative to automatically adjusting upper and lower alarm limits, a countdown timer that reflects an amount of time remaining before the expiration time ends, a first option to cancel the improvement alarm mode, and a second option to extend the improvement alarm mode.

18. The medical device of claim 17, wherein the memory device stores additional instructions which, when executed by the at least one processing device, further cause the at least one processing device to:
upon selection of the second option to extend the improvement alarm mode, extend the improvement alarm mode for a preset time period.

19. The medical device of claim 13, wherein the memory device stores additional instructions which, when executed by the at least one processing device, further cause the at least one processing device to:
generate an alarm when the measurements of the physiological variable are higher than an upper alarm limit automatically adjusted by the improvement alarm mode or when the measurements of the physiological variable are lower than a lower alarm limit automatically adjusted by the improvement alarm mode.

20. The medical device of claim 13, wherein the physiological variable is non-invasive blood pressure, blood oxygen saturation (SpO2), respiration rate, pulse rate, or temperature.
